SEQUENCE LISTING (2 sequences)

SEQ ID NO: 1 - RNA, 22 nt, Homo sapiens
uggagagaaaggcaguuccuga

SEQ ID NO: 2 - RNA, 22 nt, Homo sapiens
ucccccucagaugaucucucca

(19) United States
(12) Patent Application Publication
(10) Pub. No.: US 2022/0016157 A1
(43) Pub. Date: Jan. 20, 2022

(54) METHOD FOR PREVENTING ORAL CANCER

(71) Applicant: Research Institute at Nationwide Children's Hospital, Columbus, OH (US)

(72) Inventors: Yu Zhou, Beijing (CN); Xiaobing Guan, Beijing (CN); Li Chen, Beijing (CN)

(21) Appl. No.: 17/371,765

(22) Filed: Jul. 9, 2021

Related U.S. Application Data
(63) Continuation of application No. 16/680,374, filed on Nov. 11, 2019, now abandoned.
Application No. 16/680,374 is a continuation of application No. PCT/CN2018/086347, filed on May 10, 2018.

(30) Foreign Application Priority Data
May 11, 2017 (CN) 201710330847.X

(51) Int. Cl. A61K 31/7105; A61P 35/00; C12N 15/113

(52) U.S. Cl. CPC A61K 31/7105; A61P 35/00; A61K 9/0014; C12N 15/1137; C12N 15/1136

(57) ABSTRACT

Disclosed are a method for preventing and treating oral cancer with an exosome carrying miR-185, and a pharmaceutical composition which contains a modified salivary exosome and is used for preventing and treating oral cancer.

### CROSS-REFERENCE RELATED APPLICATIONS

This application is a continuation application of Ser. No. 16/680,374, filed Nov. 11, 2019, which is a continuation application of International Patent Application No. PCT/CN2018/086347, filed May 10, 2018, which claims priority to and the benefit of Chinese Patent Application No. 201710330847.X, filed May 11, 2017, the entire contents of each of which are incorporated herein by reference.

### SEQUENCE LISTING

The instant application contains a Sequence Listing which has been submitted electronically in ASCII format is hereby incorporated by reference in its entirety. Said ASCII copy, created on Sept. 30, 2021, is name 106887-8023_SL.txt and is 4,096 bytes in size.

### TECHNICAL FIELD

The invention relates to a method for treating leukoplakia and preventing oral cancer, and in particular to a method for preventing transformation of leukoplakia into oral cancer, comprising administering to a subject an exosome introduced with miR-185.

### BACKGROUND

Oral cancer is one of the 10 most common cancers in the world, accounting for 80% of head and neck cancers. There are about 5 million patients with oral cancer worldwide, among which oral squamous cell carcinoma (OSCC) is the most common, with a five-year survival rate of about 35-57%, and there are about 130,000 oral cancer patients die each year [1-2]. Oral cancer mainly occurs in middle-aged and elderly people. Despite advances made in diagnostic techniques, surgery, chemotherapy, and radiation therapy in recent years, unfortunately, the five-year survival rate of patients is still hovering around 50%.

Oral precancerous lesions refer to certain clinic (i.e. histological) changes of oral and maxillofacial region that have a cancerous tendency, including leukoplakia, erythema, lichen planus, discoid lupus erythematosus, submucous fibrosis, papilloma, chronic ulcer, mucosal melanoplakia and pigmented nevus, etc., of which oral leukoplakia is recognized as one of the most typical precancerous lesions in oral streak diseases, and its canceration rate is as high as 10-36%.

Oral leukoplakia (OLK), also known as oral mucosal leukoplakia, was first named by the Hungarian dermatologist Er no Sohuimmer in 1887, and refers to white or grayish keratinized abnormal lesions that occur on the oral mucosa. Oral mucosal leukoplakia is commonly seen in middle-aged and elderly people, and mostly occurs on the mucous membranes such as lips, cheeks, tongue and palate, etc. It generally has no subjective symptoms and presents milky white plaque, with surface smooth, flat or slightly higher than normal mucosa at the beginning. Leukoplakia can experience several to more than ten years from precancerous lesions to oral cancer, and the process of canceration is also multi-stage and multi-step, which must undergoes the evolution of hyperplasia—squamous metaplasia—mild, moderate, severe abnormal hyperplasia—carcinoma in situ—invasive carcinoma ^{[3-4]}, and most of the oral leukoplakia can be in a long-term benign state without canceration, only a small number of oral leukoplakias undergo precancerous lesion, precancerous state and develop into cancer. In recent years, the incidence of oral cancer tends to increase and occur significantly among youngers. Although the surgery, radiation therapy and chemotherapy techniques for oral cancer are progressing, the five-year survival rate is still less than 50%, among which the five-year survival rate for tumor-limited patients is approximately 80%, and that for metastatic patients decreased to 20% ^{[5]}.

The molecular biological mechanism of leukoplakia transformation into cancer is not well understood. Studies have shown that abnormal epithelial-mesenchymal transition (EMT), angiogenesis, apoptosis, and autophagy are closely related to malignant transformation of oral mucosal leukoplakia ^{[6-9]}.

EMT is a phenomenon in which epithelial cells transform into mesenchymal cells under physiological or pathological conditions. During this process, epithelial cells lose cell polarity and cell contact inhibition, and obtain the mobility of mesenchymal cells. Tumor cells can obtain the ability of cell invasion and metastasis by activating EMT, including the ability to obtain certain stem cell characteristics and apoptosis escape. EMT is the primary critical step in tumor invasion and metastasis. The mechanism of EMT formation is still unclear, but involves multiple signaling pathways, among which the activation of PI3K/AKT pathway is the key to the activation of EMT ^{[10]}. During EMT activation, epithelial cells gradually lose their cellular markers, such as E-cadherin and Z0-1 ^{[11]}, and express mesenchymal cell markers, such as vimentin, N-cadherin or fibronectin, etc. ^{[12,13]} Epithelial cells differentiate into fibroblast-like cells through a series of cytoskeletal recombinations and allosteries, and obtain biological properties that facilitate cell migration. In addition to acquiring the ability of cell metastasis and invasion, EMT is also closely related to the formation of tumor stem cells. Recent studies have found that TGF-β induces EMT, which can transform some epithelial cells into mesenchymal stem cells^{[}14]. It can be seen that the activation of EMT helps cells acquire the properties of tumor stem cells, so EMT is closely related to the tumor neogenesis. Studies have also confirmed that breast cancer cells can increase tumorigenicity and “stem cell transformation” of tumor cells through high expression of VEGF-A and angiogenesis during EMT ^{[15]}. Neonatal tumor stem cells promote malignant metastasis of tumors and make tumor cells lose their sensitivity to radiation and chemotherapy. Therefore, the generation of tumor stem cells or the maintenance of stem cell characters is the main cause of treatment failure and tumor recurrence.

Autophagy is one of the forms of programmed cell death, which has attracted much attention in recent years. Autophagy is an adaptive response to exogenous stimuli, including nutrient deficiency, cell density load, hypoxia, oxidative stress, and infection. Autophagy can act as a defense mechanism to remove damaged organelles and metabolites in the cytoplasm, reorganize at the subcellular level, and protect damaged cells, and as a cell death program to induce cell autonomic death ^{[16]}. The changes in autophagy activity are related to the occurrence and development of tumors. Autophagy can affect tumor progression from multiple levels, including tumor cell apoptosis, angiogenesis and chemotherapy resistance ^{[17]}. Studies have shown that EMT can deeply influence T cell-mediated immune monitoring of cancer cells: during EMT, tumor cells acquire hCD24/CD44+/ALDH-stem cell population, escape from cytotoxic T cell-mediated autophagy, thus making tumors acquire chemotherapy resistance. On the contrary, autophagy can regulate the process of EMT through the expression of adhesion molecules^{[18]}. Studies have found that the deficiency of autophagy can induce the production of EMT and promote the metastasis of gastric cancer cells ^{[19]}. However, it has been reported that activation of autophagy can induce EMT and promote intrahepatic proliferation of liver cancer cells ^{[20]}. Thus, the activity of autophagy is completely different in different tumors or even in different stages of development of the same tumor.

The EMT and malignant processes of oral mucosal leukoplakia are associated with precise regulation of molecules including microRNAs ^{[21]}. MicroRNA is a group of uncoding RNAs with a length of 18-25 nucleotide single chains, which is complementary and paired with the 3′-uncoding translation region (3′-UTR) of the target gene RNA (mRNA) and modifies the target gene at a post-transcriptional level to regulate gene expression. MicroRNAs are involved in a variety of biological processes, including growth, differentiation, apoptosis, and proliferation by modulating their target genes^{[22]}. Studies have found that the expressions of miR-10b, miR-708 significantly increased in oral mucosal leukoplakia tissues with epithelial abnormal hyperplasia, while the expressions of miR-99b, miR-145 and miR-181c were significantly down-regulated ^{[23]}. The expression level of microRNAs in tissues is related to the cytopathological characteristics. The expressions of miR-21, miR-345 and miR-181b in oral cancer are significantly higher than those in oral mucosal leukoplakia and normal mucosal tissues. However, the expressions of miR-21, miR-181b significantly increased in oral mucosal leukoplakia cells with increased mitosis, high nucleocytoplasmic ratio and dark staining. MiR-345 is highly expressed in oral mucosal leukoplakia with increased nucleus or increased volumes and high nucleocytoplasmic ratio. The expressions of microRNAs are also associated with histopathological progression. In the study of progressive and non-progressive development of oral mucosal leukoplakia, it was found that the expressions of miR-21, miR-345 and miR-181b continued to increase with the development of the disease ^{[}23-25].

On the whole, in the process of occurrence and development of oral cancer, there are obvious abnormal expressions of microRNAs, and the expression trends and effects are different ^{[26-32]}.

At present, the clinical treatment of oral leukoplakia mainly adopts chemical drugs, traditional Chinese medicine, microwave, cryotherapy and other treatment methods, among which systemic or local drug treatment is more widely used. However, drug treatment is only applicable to: (1) large area or multiple lesions; (2) the lesions located in sensitive anatomical area and cannot be removed; (3) recurrent lesions after multiple resections; (4) patients whose physical conditions are not suitable for surgical resection. For patients with a higher risk of canceration, if the lesions are limited and the surgical operation is feasible, surgical resection is still the first choice for treatment. Research has shown that up to now, there is no effective clinical method to prevent the malignant development of oral leukoplakia ^{[33]}. Once the oral leukoplakia becomes malignant and transforms into oral cancer, the average 5-year survival rate is less than 50% ^{[34-35]}, and some treatments may disfigure or cause disability.

Therefore, people are eager to find an effective method for treating leukoplakia and preventing the transformation of leukoplakia to oral cancer, so as to fundamentally prevent the occurrence of oral cancer. The inventors of the present invention find that exosomes carrying miR-185 can effectively treat oral leukoplakia, prevent the transformation of leukoplakia to abnormal hyperplasia and oral cancer, and prevent the occurrence of oral cancer, by local administration, which poses great clinical development and application value.

### SUMMARY OF THE INVENTION

The inventors found that by introducing miR-185 into salivary exosomes and administering to subjects, the inflammatory response, abnormal hyperplasia of oral mucosal epithelial cells, and mucosal microangiogenesis could be inhibited, and the transformation of oral leukoplakia into oral cancer could be blocked.

Therefore, in one aspect, the invention relates to:

A method for prophylaxis or prevention of the transformation of oral leukoplakia to oral cancer, comprising administering to a leukoplakia subject a therapeutically effective amount of an exosome carrying miR-185. In a preferred embodiment, the leukoplakia is leukoplakia with simple hyperplasia or leukoplakia with abnormal hyperplasia. In a preferred embodiment, the oral cancer is oral squamous cell carcinoma. In a preferred embodiment, the exosome carrying miR-185 is administered together with other drugs or methods that prevent the transformation of oral leukoplakia to oral cancer.

In one aspect, the invention also relates to a method for the treatment of oral leukoplakia, comprising administering to a leukoplakia subject a therapeutically effective amount of an exosome carrying miR-185. In a preferred embodiment, the leukoplakia is leukoplakia with simple hyperplasia or leukoplakia with abnormal hyperplasia. In a preferred embodiment, the treatment comprises reducing the area of leukoplakia or eliminating leukoplakia, alleviating leukoplakia with abnormal hyperplasia, reversing to simple hyperplasia, or converting leukoplakia to normal mucosa. In a preferred embodiment, the exosome carrying miR-185 is administered together with other drugs or methods for treatment of oral leukoplakia.

In one aspect, the invention relates to use of an exosome carrying miR-185 in the preparation of a pharmaceutical composition, kit or a pharmaceutical product for prophylaxis or prevention of the transformation of oral leukoplakia into oral cancer in a subject suffering from oral leukoplakia. In a preferred embodiment, the leukoplakia is leukoplakia with simple hyperplasia or leukoplakia with abnormal hyperplasia. In a preferred embodiment, the oral cancer is an oral squamous cell carcinoma. In a preferred embodiment, the exosome carrying miR-185 is administered together with other drugs or methods that prevent the transformation of oral leukoplakia to oral cancer.

In a preferred embodiment, the exosome carrying miR-185 as described above is administered to a subject by a topical route of administration. In a preferred embodiment, the exosome carrying miR-185 is administered to the subject by submucosal injection, topical smear, or buccal administration.

In one aspect, the present invention also relates to a method for prophylaxis of oral cancer, comprising administering to a subject a prophylactically effective amount of an exosome carrying miR-185. The exosome prevents the transformation of simple mucosal leukoplakia to leukoplakia with abnormal hyperplasia and oral cancer or prevents the transformation of leukoplakia with abnormal hyperplasia to oral cancer by one or more of the following mechanisms: inhibition of inflammation response, inhibition of oral mucosal epithelial cell abnormal hyperplasia, and inhibition of mucosal microangiogenesis.

In one aspect, the present invention also relates to the use of an exosome carrying miR-185 in the preparation of a drug for preventing oral cancer, wherein the exosome prevents the transformation of simple mucosal leukoplakia to leukoplakia with abnormal hyperplasia and oral cancer or prevents the transformation of leukoplakia with abnormal hyperplasia to oral cancer by one or more of the following mechanisms: inhibition of inflammatory response, inhibition of oral mucosal epithelial cell abnormal hyperplasia, and inhibition of mucosal microangiogenesis.

In one aspect, the present invention relates to a modified salivary exosome introduced with a prophylactically or therapeutically effective amount of miR-185. The invention also relates to a pharmaceutical composition, kit or pharmaceutical product containing the exosome for prophylaxis or prevention of the transformation of oral leukoplakia to oral cancer. In a preferred embodiment, the leukoplakia is leukoplakia with simple hyperplasia or leukoplakia with abnormal hyperplasia. In a preferred embodiment, the oral cancer is oral squamous cell carcinoma.

In one aspect, the invention also relates to the use of miR-185 or an exosome carrying miR-185 in the preparation of a drug for inhibiting the proliferation of oral cancer cells. Meanwhile, the invention also relates to a method for inhibiting the proliferation of oral cancer cells, comprising administering to a subject an effective amount of miR-185 or an exosome carrying miR-185 to inhibit the growth of oral cancer cells. In a preferred embodiment, the miR-185 or the exosome carrying miR-185 inhibits the growth and proliferation of oral cancer cells by topically administering to the subject. In a preferred embodiment, the miR-185 or the exosome carrying miR-185 is used in combination with other oral cancer therapeutic drugs or methods. Based on the discovery of the invention, the present application also relates to pharmaceutical compositions, preparations and kits containing miR-185 or exosomes carrying miR-185 for inhibiting the growth of oral cancer cells.

In one aspect, the invention relates to the use of miR-185 or an exosome carrying miR-185 in the preparation of a drug for the regulation of expression of oral cancer cell-associated proteins VEGF and AKT in a subject suffering from oral cancer. Meanwhile, the invention also relates to a method for regulating expression of oral cancer cell-associated proteins VEGF and AKT in a subject with oral cancer, comprising administering an effective amount of miR-185 or an exosome carrying miR-185 to the subject. In a preferred embodiment, the regulation includes inhibiting the expression of oral cancer cell-associated proteins VEGF and AKT. In a preferred embodiment, the miR-185 or the exosome carrying miR-185 regulate the expressions by topical administration to the subject. In a preferred embodiment, the miR-185 or exosome carrying miR-185 is used in combination with other oral cancer therapeutic drugs or methods. Based on the discovery of the invention, the present application also relates to pharmaceutical compositions, preparations and kits containing miR-185 or exosome carrying miR-185, which modulate the expression of oral cancer associated proteins VEGF and AKT.

The exosome carrying miR-185 described herein may be an exosome introduced with miR-185 by genetic engineering methods, or an exosome derived from human tissues, cells, blood or other body fluids that naturally has high copy of miR-185. In some embodiments, the exosome carrying miR-185 described herein is an artificially modified exosome that is introduced or increased with miR-185 by a genetic engineering method. In some embodiments, the exosome carrying miR-185 described herein is a naturally existing exosome purified from tissues, cells or body fluids, such as exosomes carrying high copy miR-185 that are derived from stem cells (e.g., mesenchymal stem cells) or other body fluids. Those skilled in the art will understand that conventional genetic engineering methods can be used to introduce miR-185 into an exosome or increase the copy number of miR-185 in an exosome.

### BRIEF DESCRIPTION OF THE DRAWINGS

FIGS. 1A-B shows the expressions of VEGF and AKT in OSCC cancer cells, and the experimental results showed that miR-185 regulated the transcriptions of VEGF and AKT in OSCC cells.

FIG. 2 shows that miR-185 inhibited the proliferation of cancer cells.

FIG. 3 shows the binding sites of miR-185 in AKT 3′-UTR, demonstrating that miR-185 had direct regulation on AKT transcript sequence.

FIG. 4 shows that miR-185 acted directly on the 3′-UTR region of AKT, regulating the survival of cancer cells.

FIG. 5 shows that miR-185 was expressed in the exosomes secreted by OSCC cells.

FIGS. 6A-B shows the size and concentration of exosomes isolated from OSCC cell line. It can be seen under transmission electron microscopy that the exosomes collected and purified from OSCC cells had uniform granule size and uniform morphology, and the exosomes were in the form of round or elliptical membranous vesicles which could be seen to have complete capsule after staining, and contain a low-electron dense substance with a diameter of about 100 nanometers (FIG. 6A).The analysis of the size of exosomes by NTA technology indicated that there were exosomes with diameter of 120 nm (FIG. 6B); insets: CD81, CD63 and Flotillin were exosome characteristic marker proteins.

FIG. 7 shows PH26 fluorescein-labeled exosomes carrying miR-185 entered into OSCC cells.

FIGS. 8A-B shows that exosomes carrying miR-185 changed the expressions of VEGF and AKT in OSCC. The experimental results showed that the highly expressed miR-185 in the OSCC cell line significantly inhibited the transcription of VEGF and AKT.

FIG. 9 shows the results of miR-185 in situ hybridization of oral mucosal tissues. The experiment analyzed the expression and distribution of miR-185 in oral mucosal tissues. The results found that in normal oral mucosa, strong brown-purple reaction appeared in a large number of epithelial nucleus and patina, and miR-185 expression was strongly positive; in oral mucosal leukoplakia with simple hyperplasia, leukoplakia with abnormal hyperplasia and oral cancer cases, the expression of miR-185 was significantly attenuated; in oral cancer cases, the expression of miR-185 disappeared in cancerous epithelial tissues.

FIGS. 10A-B shows the identification results of salivary exosomes and blood exosomes, in which 10A shows the identification result of salivary exosomes and 10B shows the identification result of the blood exosomes. The analysis of the size of exosomes by NTA technology showed that there were exosomes with diameter of 110-120 nm. Western blot analysis revealed that these granules expressed exosome specific structural protein CD81, CD63 or Flotillin (FIGS. 10A, B).

FIGS. 11A-C shows the matrix analysis result of salivary exosomes carrying small molecule microRNAs. FIG. 11A shows the matrix analysis result of salivary exosomes carrying small molecule microRNAs in cells of leukoplakia with simple hyperplasia tissue relative to normal mucosal tissue cells. The results showed that the content of microRNAs in the exosomes of oral mucosal leukoplakia saliva was significantly different from exosomes of healthy people, wherein the miR-185 from salivary exosomes of oral mucosal leukoplakia with simple hyperplasia significantly reduced compared with normal people. FIGS. 11B-C shows that the concentration of salivary exosomes significantly increased in the stage of leukoplakia abnormal hyperplasia, but significantly decreased after developing into oral cancer. In contrast, the concentration of blood exosomes markedly elevated in the stage of oral cancer.

FIG. 12 is a schematic diagram drawn based on the results of cell level measurement, showing that the exosome mediated intracellular transmission of miR-185 and affected the transcription inhibition of VEGF and AKT in the signaling pathway of oral precancerous lesions.

FIGS. 13A-B shows an animal experiment scheme and method diagram in the research of precancerous lesions progression delayed by exosomes carrying miR-185.

FIGS. 14A-H shows the lesion changes and pathological changes of golden hamster cheek pouch mucosa after 6 weeks of local application of DMBA. Cheek pouch changed from normal mucosa (FIG. 14A) to inflammatory state (FIGS. 14B-C) and developed to precancerous lesion (FIGS. 14D-E). Pathological changes transformed from normal mucosa (FIG. 14F) to simple hyperplasia (FIG. 14G) and abnormal hyperplasia (FIG. 14H).

FIG. 15 shows the weight changes of the hamsters. Three groups of hamsters were compared with the negative control group, *p<0.05, **p<0.01.

FIG. 16 shows the level of blood biochemical markers associated with liver and kidney function in hamsters.

FIGS. 17A-B shows the expression and counting of hamster cheek pouch mucositis cells. Expression (A) and counting level (B) of three groups of hamster cheek pouch mucositis cells at different stages, compared with the DMBA group, *p<0.05, ***p<0.001.

FIGS. 18A-B shows the counting results of simple hyperplasia and abnormal hyperplasia of hamster cheek pouch mucosa. Counting of simple and abnormal hyperplasia of hamster cheek pouches in the three groups, *p<0.05, **p<0.01, ***p<0.001.

FIGS. 19A-D shows the immunohistochemical staining of hamster cheek pouch mucosa. Expressions of CD31, PCNA, COX2 between groups (19A); The results of microvascular density (MVD) calculated by CD31-labeled vascular endothelial cells (19B), average optical density (AOD) of epithelium calculated by PCNA staining (19C), positive cells counted by COX2 staining (19D). The immunohistochemical staining of COX2, PCNA, CD31 in three groups of hamsters at different stages, compared with DMBA group, *p<0.05, **p<0.01, ***p<0.001.

FIGS. 20A-C shows the expression levels of inflammatory factor IL-β, IL-6, IL-10 in the serum of hamster. Expression levels of cytokine IL-6, IL-β, IL-10 in the serum of three groups of hamsters at different stages, compared with DMBA group, *p<0.05, **p<0.01.

FIG. 21 shows the expression of inflammatory factor proteins in the cheek pouch tissues.

### DETAILED DESCRIPTION OF THE INVENTION

### Definitions:

“Precancerous lesion” as described herein refers to a type of lesion which is not a cancer itself but is more susceptible to transforming into cancer. “Oral precancerous/premalignant lesion (OPL)” refers to oral lesions that have morphological changes and potential for canceration, and clinically it is more common for oral epithelial precancerous lesions, such as leukoplakia, erythema, lichen planus, discoid lupus erythematosus, submucous fibrosis, papilloma, chronic ulcer, mucosal melanoplakia and pigmented nevus, as commonly observed in clinic.

“Oral leukoplakia” as described herein is a white-based lesion occurring on the oral mucosa, and cannot be wiped off or diagnosed as other definable lesions by clinical and histopathological methods, and it belongs to the category of precancerous lesions or Potentially Malignant Disorders (PMD), excluding simple hyperkeratosis that can be resolved after local factors such as smoking and local friction are removed. The oral leukoplakia of the invention is also abbreviated as leukoplakia.

Oral leukoplakia can be divided into leukoplakia in the state of simple hyperplasia and leukoplakia with abnormal hyperplasia according to its histopathological manifestations. In the present invention, the former is called as leukoplakia (simple hyperplasia), simple hyperplasia leukoplakia or the simple hyperplasia stage of leukoplakia (these terms are used interchangeably). Pathological manifestations are as follows: epithelial hyperplasia, with excessive orthokeratosis or excessive parakeratosis, or both (appeared as mixed keratinization); epithelial simple hyperplasia is a benign lesion, which is characterized by epithelial excessive orthokeratosis, distinct granule layer and thickened spinous layer, with no atypical cells. The epithelial spikes can be elongated and thickened, but are still neat and the basement membrane is clear. The lamina propria and submucosa are infiltrated with lymphocytes and plasmacytes. For leukoplakia with abnormal hyperplasia or the abnormal hyperplasia stage of leukoplakia, the malignant potential increases with the extent of epithelial abnormal hyperplasia. The histopathological changes of epithelial abnormal hyperplasia are: the polarity of epithelial basal cells disappears; more than one basal-like cell appears; the proportion of nucleoplasm increases; the epithelial spikes are droplet-shaped; the epithelial layer is disordered; the mitotic phase increases, a few abnormal mitosis are observed; mitosis appears at epithelial superficial ½; cellular polymorphism; nuclear hyperchromatism; nucleolar enlarges; cell adhesion decreases; single or agglomerated cells in the spinous cell layer keratinize; epithelial abnormal proliferation is divided into mild, moderate, and severe according to the number of characteristics mentioned above occurring.

Oral precancerous lesions, such as oral leukoplakia, are not cancers, but if they are not treated promptly and subjected to various adverse stimuli, they may develop into oral cancer. The histopathological changes of oral cancer are: in well-differentiated squamous cell carcinoma, an intercellular bridge can be seen between cells, and a layered keratin can be seen in the center of the cancer nest, which is a keratinized or cancerous bead. The poorly differentiated squamous cell carcinoma has no keratinized bead formation, and even no intercellular bridge. The tumor cells show obvious atypia and more mitotic phases.

The methods currently available for the treatment of oral leukoplakia include: surgical resection, laser, cryotherapy, photodynamic therapy; the drug treatment includes: vitamin A, 13-cis retinoic acid, isotretinoin, acitretin, lycopene, fenretinic acid, retinoic acid, retinoic acid paste and other exfoliating drugs; the treatment by traditional Chinese medicine is still in the exploratory stage: such as gynostemma pentaphyllum, Zengshengping etc. When leukoplakia is transformed into oral cancer, the current conventional treatment for cancer, including surgery, radiotherapy, or chemotherapy, can be used.

“Exosome” is a subcellular bilayer membrane vesicle with a diameter of 30-150 nm, formed by a series of regulatory processes in which cells undergo “endocytosis, fusion, and efflux”, and that can secrete to the extracellular environment. It contains substances such as proteins, miRNAs, and mRNAs related to cell source. Exosomes can directly activate recipient cells through the plasma membrane receptor, and transport proteins, mRNA, iRNA and even organelles into the recipient cells, and can also carry special “information” contained in cells in different pathological states into body fluids (including saliva, blood, etc.), thus playing an important role in both physiology and pathology.

“Therapeutically effective amount” with respect to oral leukoplakia refers to the amount of exosome carrying miR-185 that can reduce or eliminate the area of the leukoplakia, or alleviate the leukoplakia with abnormal hyperplasia, or reverse to simple hyperplasia, or even convert to normal mucosa.

“Prophylactically effective amount” with respect to oral cancer refers to the amount of exosome carrying miR-185 that can achieve any or more of the following: reducing the number of leukoplakia epithelial cells, reducing or disappearing the area of leukoplakia, weakening the inflammatory response of leukoplakia, weakening the microvascular formation of mucosa, preventing the development of simple mucosal leukoplakia to leukoplakia with abnormal hyperplasia, even the progression of oral cancer, and preventing the transformation of leukoplakia into oral cancer.

### EXAMPLES

Example 1

### The Regulatory Effect of miR-185 in the Transformation of Precancerous Lesions into Oral Cancer

### Methods

(1) Immortalized oral squamous cell carcinoma (OSCC cell line) resuscitation: the frozen OSCC cells (purchased by ATCC, ATCC® CRL-1623™ Manassas, Va., USA) were rapidly melted in a 37° C. thermostatic water bath; injected into a centrifuge tube, added with culture media, and centrifuged at 1000 rpm/min for 5 minutes; supernatant was discarded and culture media was added. Cells were cultured at 37° C., 5% CO₂ under saturated humidity; after 24 hours, the cells were observed under an inverted microscope, and the culture was replaced with fresh media.

(2) Cell culture and passage: Cells were passaged when growing to 80% 90%; the cells were digested with 0.25% trypsin, pipetted, transferred to a test tube, centrifuged, and the supernatant was discarded; the culture media was added, and cells were passaged at 1:2 or 1:3, and then cultured at 37° C., 5% CO₂ under saturated humidity (in order to ensure the stability of the cell properties, the experiments were carried out using cells within 10 times of passages).

(3) Real-time fluorescent quantitative PCR (qRT-PCR) technology was used to analyze the expression level of miR-185: the miRNAs in the cells were reverse transcribed into single-stranded cDNA (purchased by Qiagen, Omniscript RT Kit-205111, Germantown, Md., USA). The SYBR Green chimeric fluorescence method was used to detect the expression level of miR-185 by qRT-PCR, and small fragment RNA U6 was used as an internal reference (purchased from Qiagen, miScript SYBR® Green PCR Kit-218073).

(4) The miR-185 analogues (purchased from Qiagen, miScript miRNA Mimic-219600) or inhibitors (purchased from Qiagen, miScript miRNA Inhibitor-219300), and negative controls (random sequence or random inhibitor, purchased from Qiagen, miScript Inhibitor Neg. control-102727), were transfected into the OSCC cell line (Lonza Nucleofector™ system) using a liposome-encapsulated transfection reagent, respectively,. Cells were collected for extraction of total mRNAs (purchased from Qiagen, RNeasy Mini kit-74104) 48 hours later. Then qRT-PCR technology was used to analyze the expression level of VEGF or AKT.

(5) MTT method was used to detect the proliferation index of cancer cells: the transfected cells of (4) were collected and inoculated in 96-well plates, and cultured for 48 hours. The proliferation index of cancer cells was detected by MTT method using Abcam MTT detection kits (Burlingame, Calif., USA).

(6) Construction of AKT luciferase reporter genes plasmid: the potential miR-185 binding sites on the AKT genes were screened by the miRBase data analysis system (microRNA.org). The full-length 3′-uncoding translation region of AKT (3′-UTR) was amplified from genomic DNA and cloned into the plasmid vector Fire-Ctx sensor lentivector (miR-selection Fire-Ctxlentivector, purchased from SBI, Palo Alto, Calif., USA). The firefly luciferase reporter gene and Cytotoxin (CTX) drug sensitive gene was located downstream. In the experiment, Fire-Ctx sensor lentivector was used as the control.

(7) Transfection and cytotoxicity detection of the plasmid: the constructed plasmid was transfected into the OSCC cell line by electroporation technology (Lonza Nucleofector™ System, Walkersville, Md., USA), and the miR-185 precursor (pre-miR-185, purchased from Exiqon, Woburn, Mass., USA) were co-transfected in the cells. To control the transfection efficiency, cells were also transfected with the pRL-CMV vector plasmid (purchased from Promega Inc.-E2261, San Luis Obispo, Calif., USA)which comprises Renilla luciferase reporter gene. In the corresponding experiment, the cells were treated with cytotoxin (CTX) for 3 to 4 days after transfection for 24 hours, and then the survival rate of the cells was measured.

### Results

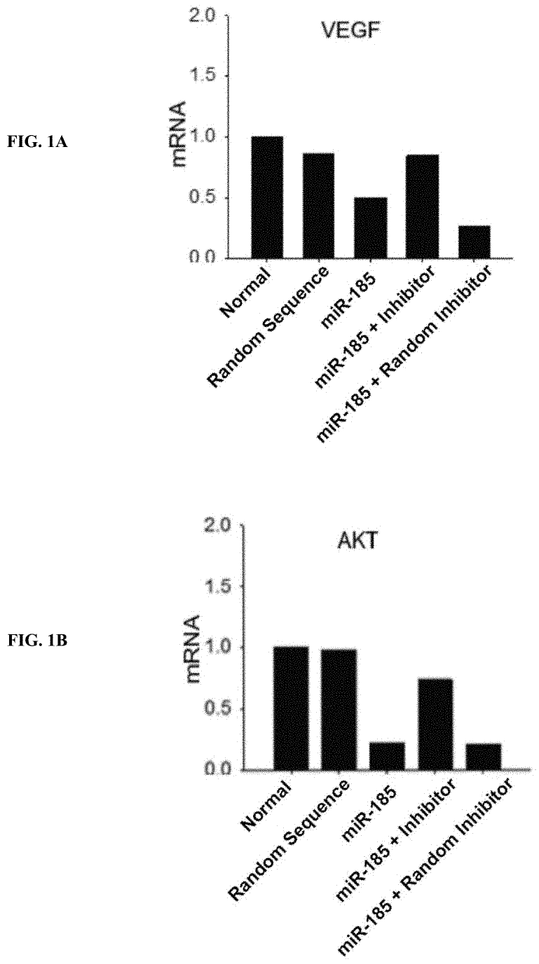

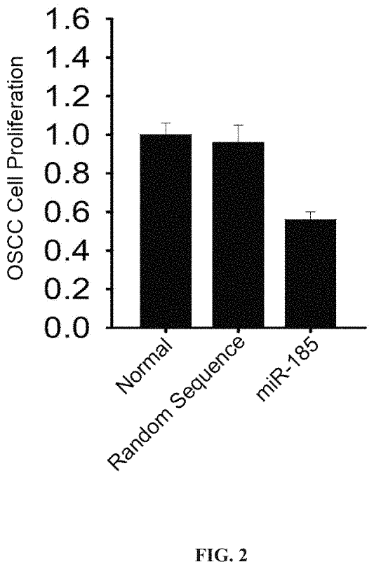

The experiment found that transfection of miR-185 (nucleotide sequence: 5′ undefineduggagagaaaggcaguuccuga 3′) analogues in OSCC cells could significantly reduce the transcription of VEGF and AKT. On the contrary, the inhibitory sequence of co-transfected miR-185 in OSCC effectively inhibited the effect of miR-185 analogues. However, the control random sequence (scramble) did not work. The experiment demonstrated that miR-185 significantly regulated the expressions of oral cancer cell-associated proteins VEGF and AKT (see FIGS. 1A-B). The experiment also found that transfection of miR-185 effectively inhibited the proliferation of cancer cells (see FIG. 2).

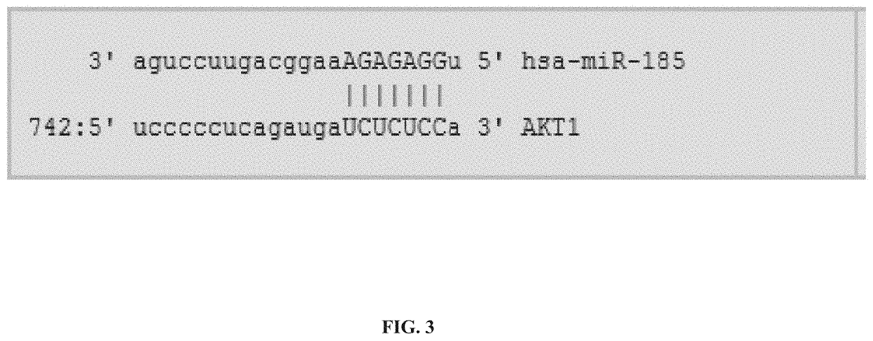

MiRBase data analysis system (microRNA.org) was used to screen the direct regulatory sites of miR-185 in AKT transcriptional sequence (see FIG. 3).

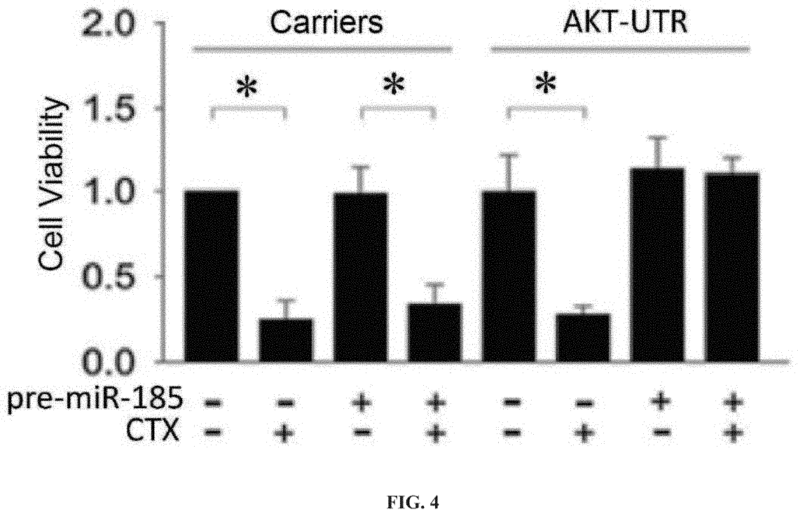

The experiment found that the Fire-Ctx AKT 3′-UTR plasmid was transfected in the OSCC cell line, and the cytotoxin (CTX) drug-sensitive gene was carried downstream. The experimental results showed that the addition of CTX toxic drugs in cell culture significantly led to a large number of cell death, but the survival rate of OSCC cell lines with high expression of pre-miR-185 was significantly increased, and there was no significant difference from the control group (see FIG. 4). The experiment suggested that miR-185 specifically acted on the 3′-UTR region of AKT, inhibited the expression of the cytotoxin (CTX) drug sensitive gene, so as to reduce cytotoxic reactions and enhance cell survival rate.

### Example 2

### Inhibition of Transcription of Recipient Cell Canceration Signaling Pathway Molecular by miR-185 Delivered by Exosomes

### Methods

(1) Isolation and purification of exosomes in the cell culture: Cultured OSCC cells (as mentioned above), after 48 hours of serum starvation, were collected and centrifuged at 2000×g for 20 minutes at 4° C. and at 10000×g for 30 minutes to remove cell debris. Exosomes were isolated by using exosomes isolation kits (Exosome isolation Kit, Cat. NO: GET301-10, Genexosome Technologies Inc., Freehold, N.J., USA), and resuspended and diluted in a volume of sterile PBS buffer.

(2) Identification of exosome characters: the exosomes obtained were observed under transmission electron microscope (TEM); the size and concentration of the exosomes were determined by NTA (Nano-trackinganalysis, ParticleMetrix GmbH, Meerbusch, Germany) analysis technology.

Western blot was used for characteristic analysis of protein markers carried by exosomes. 15% separation gel and 5% stacking gel were prepared. 40 μl of exosome suspension was mixed with 10 μl of 5X SDS loading buffer and boiled for 5 minutes and added into gel loading hole. 80V and 120V constant voltages were applied to the stacking gel and the separation gel, respectively. The gels were applied 200 mA of constant current for 1.5 hours. The proteins in the gel were transferred to a nitrocellulose membrane by wet transfer, and sealed with a sealing solution containing 5% skim milk at room temperature for 1 h. After elution with 1X TBST buffer, CD81 (1:400), CD63 (1:250) and Flottilin 1 (1:1000) monoclonal antibody (purchased from Abcam) were added and reacted overnight at 4° C. After elution again, horseradish peroxidase-labeled goat anti-rabbit secondary antibody (1:2500, Sigma St. Louis, Mo., USA) was added and gently shaken at room temperature for 1 h. After washing the membrane with 1X TBST buffer for 3 times, it was detected with a chemiluminescent substrate (ECL, purchased from Thermo Fisher Scientific, Carlsbad, Calif., USA).

(3) Real-time fluorescence quantitative PCR (qRT-PCR) technology was used to analyze the expression level of miR-185 in OSCC cells and secreted exosomes: the miRNAs in cells and exosomes were reverse transcribed into single-stranded cDNA. The SYBR Green chimeric fluorescence method was used to detect the expression level of miR-185 by qRT-PCR, and small fragment RNA U6 was used as an internal reference.

(4) Exosomes transfer between cells: exosomes carrying high copy miR-185 were extracted in conditioned medium of OSCC cells. PKH26 fluorescently labeled exosomes (PKH26Red Fluorescent Cell Linker Kit, purchased from Sigma) was added to the culture medium of OSCC cells, and after 24 hours, the exosomes fluorescently labeled with PKH26 were observed to be absorbed by OSCC cells.

(5) The effect of exosomes carrying high copy miR-185 on intracellular transmission and the regulation of transcription inhibition of signal molecules in the signaling pathway of oral precancerous lesions: OSCC target cells were cultured in the medium of exosomes carrying high copy miR-185, and the expression levels of VEGF and AKT in the target cells were detected to determine whether the canceration could be effectively reversed after the exosomes carrying mir-185 entered the receptor target cells.

### Results

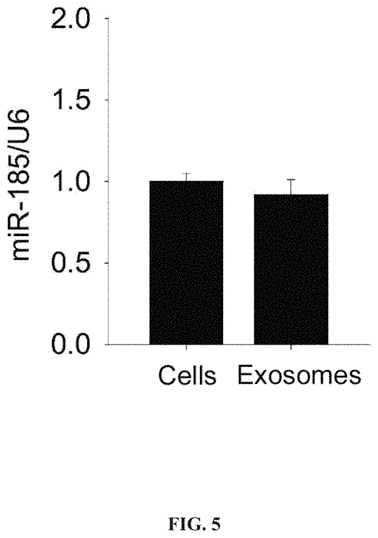

(1) It can be seen under transmission electron microscopy that the exosomes collected and purified from OSCC cells had uniform granule size and uniform morphology, and the exosomes were in the form of round or elliptical membranous vesicles which could be seen to have complete capsule after staining, and contain a low-electron dense substance with a diameter of about 100 nanometers. See FIG. 5.

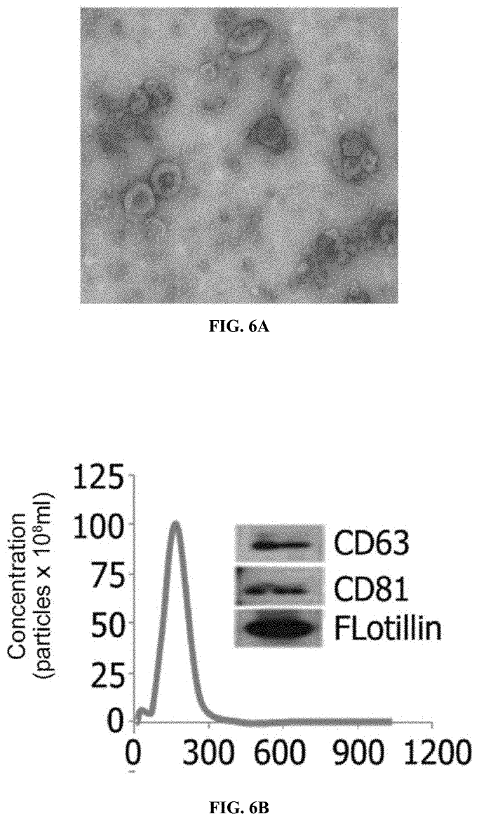

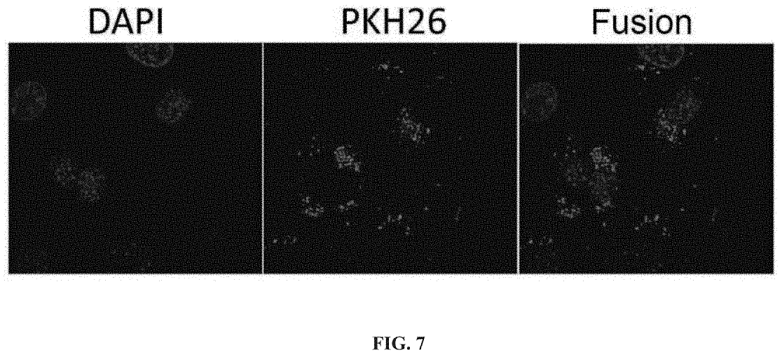

The experimental results showed that miR-185 was carried in exosomes and inhibited the transcription of VEGF and AKT. It has been reported in literatures that miRNAs are encapsulated by exosomes and released into the extracellular matrix. Early results of this experiment showed that miR-185 was expressed in the exosomes secreted by OSCC cells (see FIG. 5). According to NTA analysis, the diameter of OSCC exosomes was 120 nm (see FIG. 6). Western blot identification results confirmed that OSCC exosomes highly expressed exosomes markers such as CD81, CD63 and Flotillin (see FIG. 6).

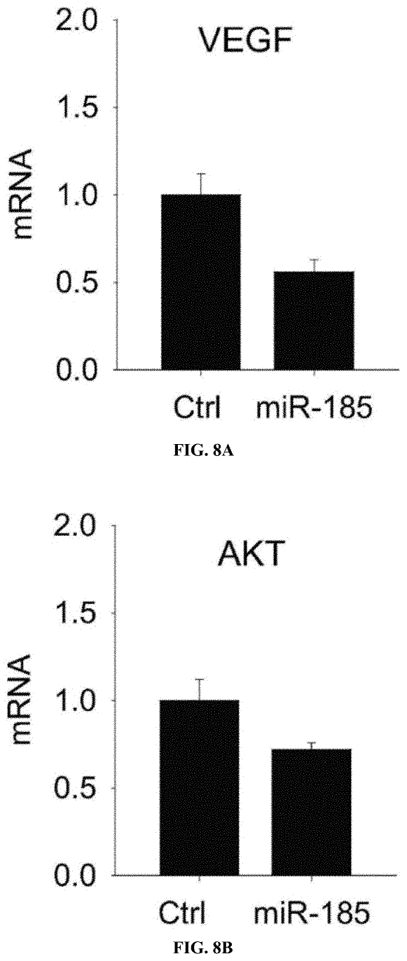

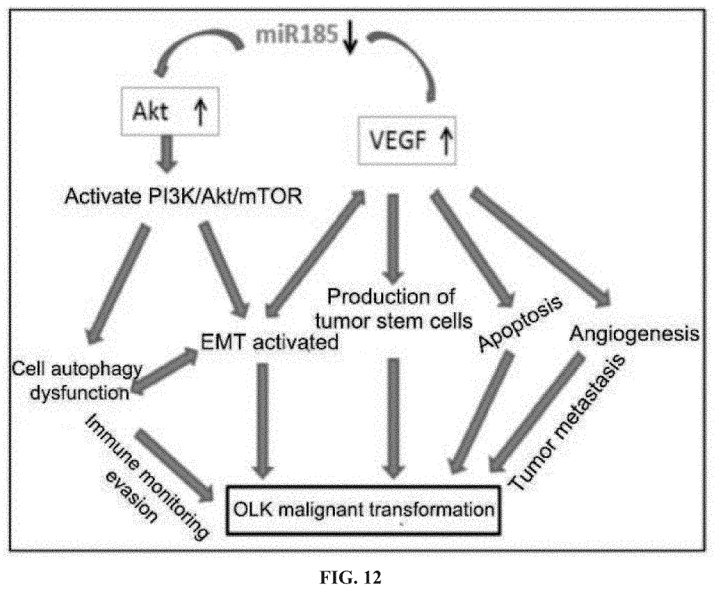

(2) miR-185 exosomes were labeled with PKH26 red fluorescent markers and then added to OSCC cell culture medium. After 48 hours, the exosomes were observed to be ingested by OSCC cells (see FIG. 7). QRT-PCR results showed that OSCC overexpressed miR-185 after ingesting miR-185 (no results displayed), and significantly inhibited the transcription of VEGF and AKT (see FIGS. 8A-B). FIG. 12 is a schematic diagram based on the results of cell level measurement, showing the exosomes mediated intracellular transmission of miR-185 and affected the inhibition of VEGF and AKT transcription in the signaling pathway of oral precancerous lesions.

### Example 3

### Changes in the Expression of miR-185 in the Process of Oral Leukoplakia to Carcinogenesis

### Methods

Tissue samples from patients clinically and pathologically diagnosed as oral mucosal leukoplakia with simple hyperplasia, leukoplakia with abnormal hyperplasia and leukoplakia canceration (oral squamous cell carcinoma) and normal tissue samples were selected as studied subjects.

According to the pathological diagnosis results, samples were divided into: leukoplakia with simple hyperplasia group (N=15); leukoplakia with abnormal hyperplasia group (N=10), cancerous group, also referred to as oral cancer group (N=15).

The tissue samples of normal control group (N=8) were selected from patients who were excluded from oral mucosal disease, needed to remove some of the normal tissues for surgical treatment and were willing to provide the tissue for the study.

In situ hybridization to localize miR-185 expression

MiR-185 or control sequence probes (Exiqon Inc.) were hybridized with fixed tissue sections in 1X in situ hybridization (ISH) buffer (purchased from Exiqon Inc., Woburn, MA USA) at 55° C. for 60 minutes, followed by washing with different concentrations of SSC buffer at 55° C. Detecting probes were as follows: incubation with monoclonal ant-digoxin alkaline phosphatase antibody (1:800) (Roche, Indianapolis, Ind. USA) for 60 minutes, and then with tetranitroblue tetrazolium chloride and 5-bromo-4-chloro-3′-polyphosphate substrate (Roche, Pleasanton, Calif., USA) at 30° C. for 2 hours. Finally, sections were counterstained using Nuclear Fast Red™, mounted using Eukitt® medium (VWR, Radnor, Pa.), and examined by confocal microscopy.

### Results

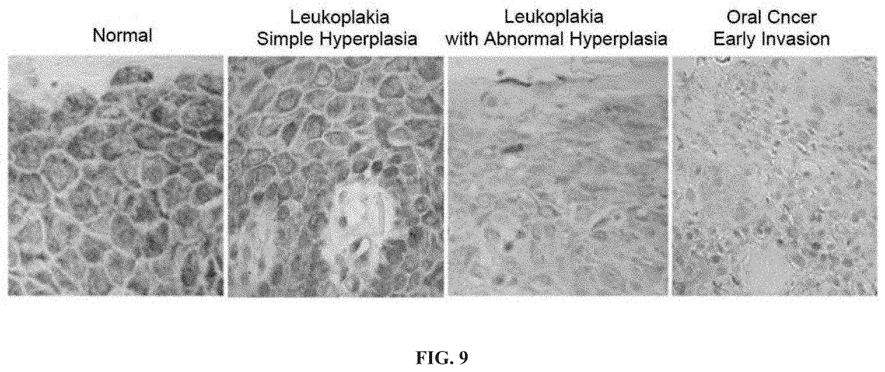

In situ hybridization experiment found that in normal group samples, the expression of miR-185 was strongly positive (purple); in leukoplakia group samples, the expression of miR-185 significantly attenuated, while in abnormal hyperplasia group and oral cancer group, a slight brown-purple reaction appeared in a small number of epithelial nucleus and patina, suggesting the expression of miR-185 was slightly positive or almost disappeared, see FIG. 9.

Recently, a variety of miRNAs that directly target EMT transcription factors and cellular structural components have been reported. The above experimental results found that the levels of miR-185 in samples of leukoplakia with simple hyperplasia group, leukoplakia with abnormal hyperplasia group and oral cancer group significantly decreased compared with normal control.

In summary, the experiment found that in the process of transformation from oral leukoplakia with simple hyperplasia to oral mucosal leukoplakia with abnormal hyperplasia and oral cancer, the PI3K/AKT-mTOR pathway was activated, EMT was occurred, and the expression of miR-185 decreased or even disappeared.

### Example 4

### Oral Salivary Exosomes or Blood Exosomes Carry miR-185 Associated with Disease Status

### Methods

Exosomes: Oral salivary exosomes were collected and purified from the oral cavity of the patients with oral mucosal leukoplakia (simple hyperplasia), leukoplakia with abnormal hyperplasia, oral cancer (oral squamous cell carcinoma) diagnosed clinically and pathologically in Example 1, as well as from normal people.

The above patients or normal people did not gargle before taking saliva, and fasted water for 1 hour. When taking saliva, the head was naturally lowered, and the saliva in the mouth was naturally spit out into a disposable tray for about 2 ml without cough. The collected saliva was immediately placed into a small centrifuge tube.

The samples were centrifuged at 4 ° C., 10,000×g for 20 minutes to remove impurities, and the supernatant was filtered twice through a 0.22 μm filter, and exosomes were isolated by using an exosome isolation kit (Cat. NO: GET200-10, Genexosome Technologies Inc., Freehold, N.J., USA), resuspended in a volume of sterile PBS buffer and diluted.

### 1. Identification of Salivary Exosomes and Blood Exosomes

### (1) Morphological Observation of Exosomes

10 μl of exosome suspension was dripped onto a copper mesh with a pore diameter of 2 nm and placed at room temperature for 10 minutes. Liquid from the side of the filter was blotted with filter paper. To the sample was added 30 μl of 3% phosphotungstic acid solution to allow counterstaining at room temperature for 5 minutes. The counterstain was blotted with filter paper. The copper mesh was dried at room temperature, placed in a sample chamber of a transmission electron microscope to observe the morphology of exosomes and photographed.

### (2) Identification of Salivary Exosomes and Blood Exosomes

The size and concentration of exosomes were detected by NTA technology.

### (3) Analysis of Exosome-Specific Structural Proteins

15% separation gel and 5% stacking gel were prepared. 40 μl of exosome suspension was mixed with 10 μl of 5X SDS loading buffer and boiled for 5 minutes. The mixture was added to gel loading holes and run at 80V of stacking gel constant voltage, 120V of separation gel constant voltage, and 200 mA of constant current for 1.5 hours. The proteins in the gel were transferred to a nitrocellulose membrane by wet transfer, and sealed with a sealing solution containing 5% skim milk at room temperature for 1 h. After elution with 1X TBST buffer, CD81 (1:400), CD63 (1:250) and Flottilin (1;1000) (Abcam) monoclonal antibody were added and reacted overnight at 4° C. After elution again, horseradish peroxidase-labeled goat anti-rabbit secondary antibody was added and gently shaken at room temperature for 1 h. After washing the membrane with 1X TBST buffer for 3 times, it was detected with a chemiluminescent substrate (ECL, Thermo Fisher Scientific).

### 2. Matrix Analysis of Salivary Exosomes Carrying Small Molecule MicroRNAs

MicroRNeasy Plus kit (Qiagen, Valencia, Calif. USA) was used to extract total RNAs from salivary exosomes, and miScript II RT kit (Qiagen) was used for reverse transcription according to the manufacturer's instructions. The transcripts obtained by microRNA matrix analysis were verified by qRT-PCR according to the manufacturer's instructions. QRT-PCR was normalized to U6snRNA primers.

3. The changes of salivary exosomes and blood exosomes concentration during the development of oral leukoplakia to canceration were detected by NTA technology.

### Results

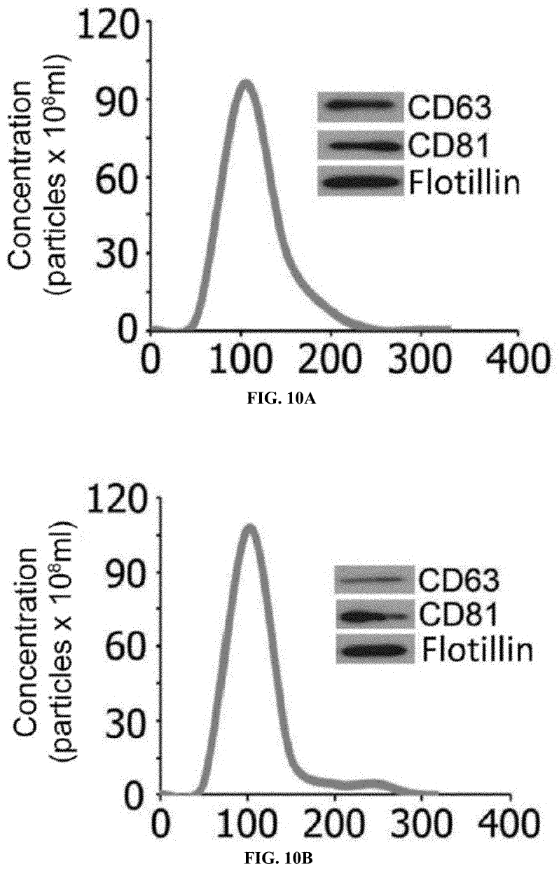

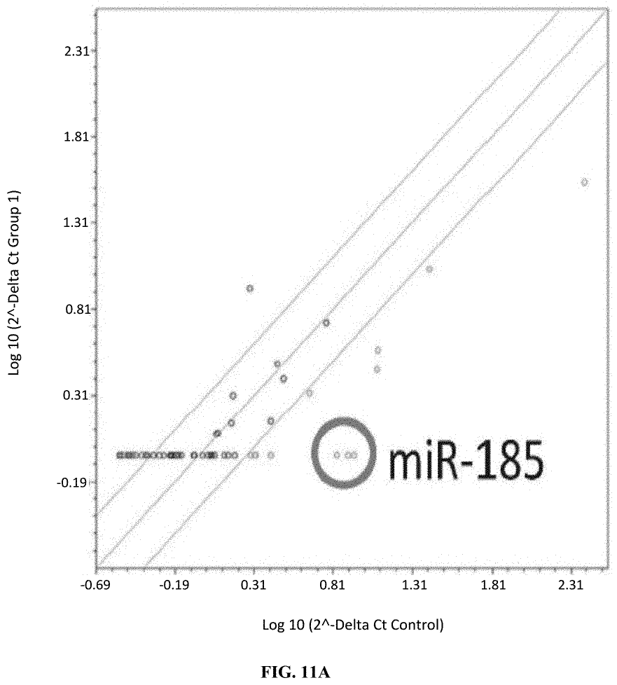

The size of salivary-derived exosomes or blood-derived exosomes was found to be 110-120 nm by NTA technology (FIGS. 10A, B). These granules were found to express the exosome-specific structural proteins CD81, CD63 or Flotillin as detected by Western blot analysis. See FIGS. 10A, B.

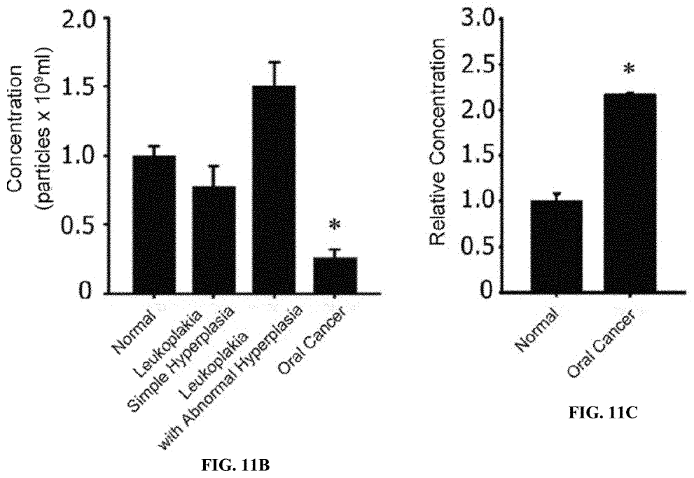

2. Through the microRNA matrix, we found for the first time that the content of miR-185 from salivary exosomes of patients with leukoplakia with simple hyperplasia was significantly lower than exosomes from normal people. See FIG. 11A.

3. The concentrations of salivary exosomes in patients with leukoplakia with simple hyperplasia, leukoplakia with abnormal hyperplasia, and oral cancer were significantly different. The concentration of salivary exosomes in patients with leukoplasia accompanied by abnormal hyperplasia significantly increased, while the concentration significantly decreased after canceration. See FIG. 11B. In contrast, the concentration of blood exosomes in patients with canceration significantly increased. This finding indicated that the concentration of salivary exosomes was closely related to the development of the disease, and the blood exosomes and salivary exosomes showed an opposite secretion trend (FIGS. 11B, C).

### Example 5

### Exosomes Carrying miR-185 Blocked the Progression of Precancerous Lesions

### Methods

### (1) Reagents and Preservation

The reagents used in this experiment and the following experiments were prepared and stored as follows: 0.5 g of dimethylbenzanthracene (DMBA) was dissolved in 50 ml of acetone and 50 ml of liquid paraffin to prepare a 0.5% DMBA solution, which was stored at room temperature in dark. The exosomes carrying miR-185 were all mesenchymal stem cell-derived exosomes containing high copy miR-185 and purchased from GenExsomeTechnology under the trade name GET MSCEXO101-1 ug. The exosome particle concentration of the exosome solution carrying miR-185 was 2×10¹¹ particles/ml. The solution was stored at −80° C., and transferred to 4° C. 24 h before use.

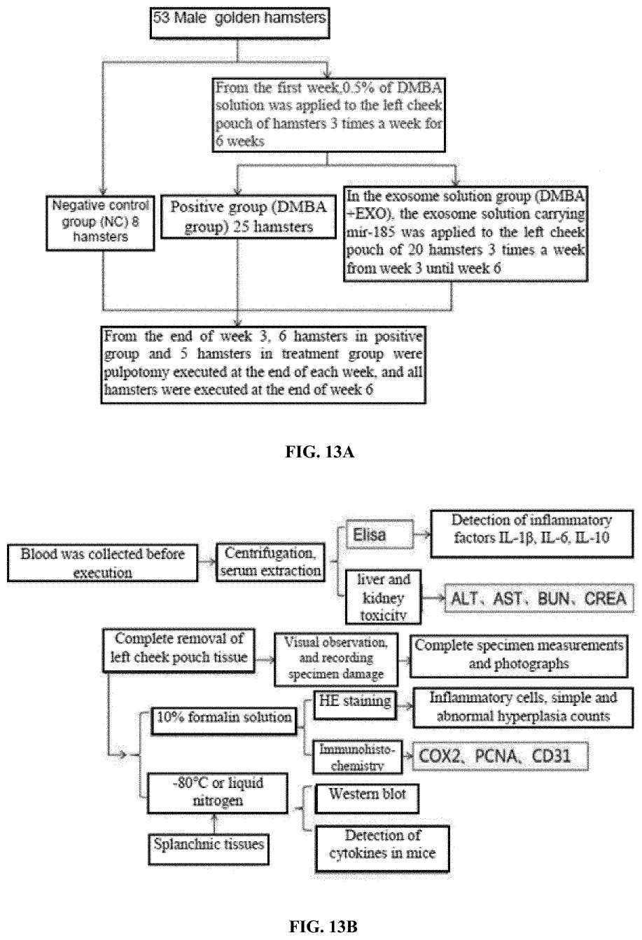

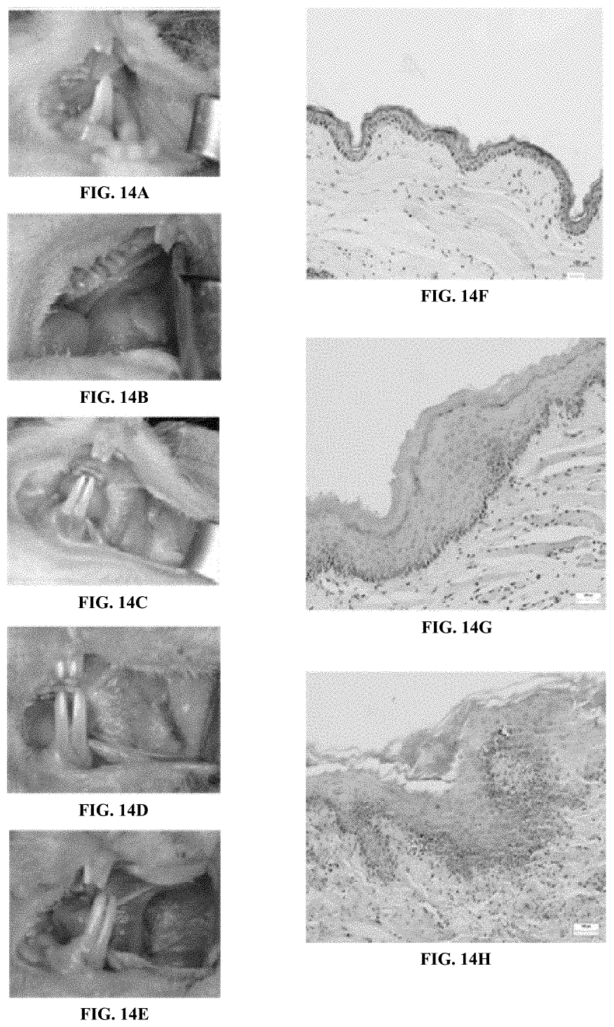

(2) SPF grade 7-week-old male Syrian golden hamsters (Beijing Vital River Laboratory Animal Technology Co., Ltd.), with an average weight of 115 g, were selected. Animals were housed at temperature 24-26° C., humidity 40-60%, 12-14 hours light on. After one week of adaptive feeding, 53 hamsters were randomly divided into 3 groups. There were 8 hamsters in negative control group (NC), 25 hamsters in positive control group (i.e., positive control group given dimethylbenzanthracene, abbreviated as DMBA group), and 20 hamsters in topical application of high-copy miR-185 exosome solution group (DMBA+EXO group, also known as treatment group). The negative control group was not treated with drugs during the whole experiment, the other two groups were applied with 0.5% dimethylbenzanthracene (DMBA) solution in the left cheek pouch from the first week, and 3 times a week until the end of the experiment. The positive control group with 25 hamsters no longer had other treatments; in the treatment groups with 20 hamsters, exosome solution was applied at the same position as DMBA 3 times a week from week 3 until the end of week 6. From the end of week 3, 6 hamsters in DMBA group and 5 hamsters in DMBA+EXO group were pulpotomy executed at the end of each week, and the remaining hamsters were executed at the end of week 6. During the experiment, the health and disease conditions of hamsters were observed and recorded, and the body weight was recorded weekly. The experiment was carried out in accordance with the technical route in FIG. 13A and the experimental method in FIG. 13B.

(3) Application method: A paint brush was dipped into the liquid, squeezed excess liquid, and applied to the center of the cheek pouch of the left side of the hamster. The application was done by circular motion in the same direction. The length and shape of the brush were adjusted by quantitative test to determine the amount of each application to be 100 μl. The exosome solution containing miR-185 and DMBA solution were applied at an interval of 4 h. The animals were fasted water for 2 hours after the application.

(4) Extraction and preservation of serum: whole blood was collected and stored in an EP tube before executing the hamsters, placed at room temperature for 30 min, and then the plasma and serum were separated by centrifugation at 4° C., 3000×g for 10 min. The serum was extracted and stored at −80° C.

(5) Liver and kidney functions: commercial kits were used for detection. The kits were purchased from InTec Products, Inc. (Xiamen), and alanine aminotransferase (ALT) was detected by UV-lactate dehydrogenase method, aspartate aminotransferase (AST) was detected by UV-malate dehydrogenase method, creatinine (Scr) was detected by enzymatic method, urea nitrogen (BUN) was detected by UV-glutamate dehydrogenase (UGDH) method, and the experiment was conducted in strict accordance with the instructions of the kits.

(6) Embedding section: hamster cheek pouch tissues were fixed in 10% formalin solution for 24 hours, then taken out, cut into strips of about 3-5mm, rolled into tube shapes, fixed with steel needle, dehydrated by automatic dehydration machine, removed steel needle and embedded in paraffin wax. Each sample was continuously cut into 21 sheets of 5 μm sections, and the 1st, 10th, and 20th sheets were subjected to HE staining, and the 2nd, 11th, and 21st sheets were subjected to immunohistochemical staining.

(7) HE staining: the slides were baked in an oven at 65° C. for 1 h, routinely dewaxed to water, rinsed with tap water for 2 min, rinsed with tap water after rinsed with hematoxylin stain for 4 min, differentiated in differentiation solution for 2s, returned to blue in bluing liquid for 4 s, soaked in tap water for 5 min, dyed in eosin stain for 40 s, rinsed with tap water for 30 s, dehydrated into xylene, and sealed with neutral resins. Infiltration of inflammatory cells (could be determined as lymphocytes and neutrophils by morphology) in the mucosa lamina propria and submucosa was observed under 400× microscope. 3-10 areas with more inflammatory cells in the field of view were selected on each slide and cells were counted under a 200× microscope. Simple hyperplasia was characterized by an increase in the number of cells, an obvious epithelial granule layer and acanthosis, and no atypical cells; Abnormal hyperplasia, according to the diagnostic criteria of WHO, comprises disappearance of epithelial basal cell polarity, appearance of more than one basal-like cells, increase in proportion of nucleoplasm, droplet shape of epithelial spike, disorder of epithelial hierarchy, increase in nuclear mitosis, observation of a few abnormal nuclear mitosis figures, occurrence of mitosis in ½ epithelial superficial, pleomorphism of cells, hyperchromatic nuclei, enlarged nucleoli, decrease of cell adhesion, and keratinization of single or clustered cells in the layer of spinous cells. The total number of simple and abnormal hyperplasia of samples was recorded in strict accordance with the criteria.

(8) Immunohistochemistry experiments: The sections were baked in an oven at 65° C. for 1.5 h, routinely dewaxed to water, washed with PBS buffer, microwave-repaired with 0.01 mol/L sodium citrate buffer, placed at room temperature, and the slides were placed in a wet box after washing, sealed at room temperature with 3% of hydrogen peroxide for 15 min, washed, incubated with 10% of goat serum at 37° C. for 1 h to block the antigen. The excess serum was discarded, and the primary antibodies were added dropwise at concentrations of: 1:200 for anti-CD31 antibody, 1:30000 for anti-PCNA antibody, 1:1000 for anti-COX2 antibody. All antibodies were purchased from Abcam. The blank control used PBS instead of primary antibodies, and placed at 4° C. overnight. The next day, the slides were taken out and rewarmed at room temperature for 1 h, washed with PBS buffer, added secondary antibodies dropwise, incubated at 37° C. for 0.5 h. DAB (diaminobenzidine) was added dropwise after washing, and slides were observed under microscope for color development and timing, water washed to stop color development, counterstained with hematoxylin, blued, dehydrated, transparentized, and sealed with neutral resins. The expression of PCNA protein was located in the nucleus. Each slide was selected 3-5 epithelial hyperplasia (simple hyperplasia, abnormal hyperplasia) area under 100× microscope of. Optical density was analyzed by Image pro plus software, and integrated optical density value (IOD) was recorded. The expression of COX2 protein was located in the nuclear membrane, and 2-5 areas with dense inflammatory cells were selected under the 100× microscope, and the positive cells with the nuclear membrane appeared yellowish-brown or chocolate brown were counted; the CD31 protein was expressed in the endothelial cell membrane. According to the Weidner method, areas with dense microvessels (less than 8 red blood cells in diameter) were selected under the 100× microscope, and the number of microvessels labeled with CD31 was counted under 400× microscope. The mean value was the MVD value (microvascular density). The above data recorded above was compared among groups.

(9) Enzyme-linked immunosorbent assay (ELISA): commercial hamsters IL-6, IL-1β, IL-10 ELISA kits under the brand name MyBioSource (San Diego, Calif., USA) were used to detect these cytokines. The required plates were taken out from aluminum foil bag after balancing at room temperature for 20 min, blank control hole, standard hole and sample hole were set up. The blank control hole was added 50 μl of sample diluent, the standard hole was added 50 μl of different concentration of standards, and the sample hole was added 50 μl of serum. Each hole was added 100 μl of horseradish peroxidase (HRP)-labeled antibodies, sealed with microplate sealers, incubated in an incubator at 37° C. for 60 min, discarded the liquid, and dried on absorbent paper. Each hole was filled with cleaning solution, placed for 1 min, removed the cleaning solution, and dried on absorbent paper. The plates were repeatedly washed for 5 times, and each hole was added 50 μl of substrates A, B, respectively, incubated at 37° C. in the dark for 15 min, added 50 μl of stop solution, and the OD values of each hole were measured at 450 nm wavelength within 15 minutes. R² value and protein concentration of cytokines were calculated by formula and compared among groups.

(10) Mucosal protein detection: total cheek pouch mucosal proteins of the three groups of hamsters at the end of three weeks (acute inflammation period), and total cheek pouch mucosal proteins of the DMBA+EXO group at the end of six weeks were extracted (totally four groups). According to the requirements of Proteome Profiler Array Mouse Cytokine Array Panel A kits (brand R&D) instructions, 2 ml of sealing solution was added into each hole of the four-hole plates for sealing. The four membranes were placed in a four-hole plate, incubated in a shaking bed for 1 hour. The samples were prepared, and proteins were added into each test tube. The volume was adjusted to 1.5 ml by diluent, and each sample was mixed with the 15 μl of dissolved antibody, and incubated at room temperature for 1 h. The sealing solution in the four-hole plate was removed, and sample antibody mixture was added and incubated in a shaker at 4° C. overnight. The next day, the membrane was taken out and washed in the shaker for 10 min for three times. The four-hole plates were washed, and 2 ml of diluted streptavidin-HRP was added to each hole. Four membranes were placed in four-well plates and incubated at room temperature for 30 min. The membranes were washed, placed in the cassette with number up, evenly added 1 ml of developer dropwise, incubated for 1 min, developed, and exposed. The different protein sites of each membrane were observed and the gray values were analyzed and compared among groups.

(11) Statistical methods: statistical analysis was performed using SPSS 20.0 statistical software. The data was represented by mean±SD. One-way ANOVA parameter test was used and LSD was compared pairwise; simple and abnormal hyperplasia counts were represented by IQR. Rank-sum test was used and Mann-Whitney was compared pairwise with a=0.05 as the inspection level, *p<0.05, **p<0.01, ***p<0.001.

### Results

(1) Lesion changes and pathological changes: healthy mucous membranes were light pink, smooth, thin, continuous, and the submucosal blood vessels are clearly visible (see FIG. 14A). Inflammatory stage was from the beginning of the second week to the end of the third week, with mucosal hyperemia, edema and yellow liquid-like inflammatory exudation (see FIG. 14B). As the exudation increased, it condensed into a block which could be wiped off, and it was easy to bleed when the block was wiped off (see FIG. 14C). The mucous membranes were gradually crusted and had good elasticity. At the beginning of the fourth week, the mucosal elasticity gradually decreased, and keratosis appeared in some areas (see FIG. 14D). Precancerous stage was from the beginning of the fifth week to the end of the sixth week, during which the mucous membranes were rough, white and slightly thickened, and leukoplakia was partially seen (see FIG. 14E). The histopathological changes observed under light microscope gradually changed from normal mucosa (see FIG. 14F) to simple hyperplasia (see FIG. 14G) and abnormal hyperplasia (see FIG. 14H).

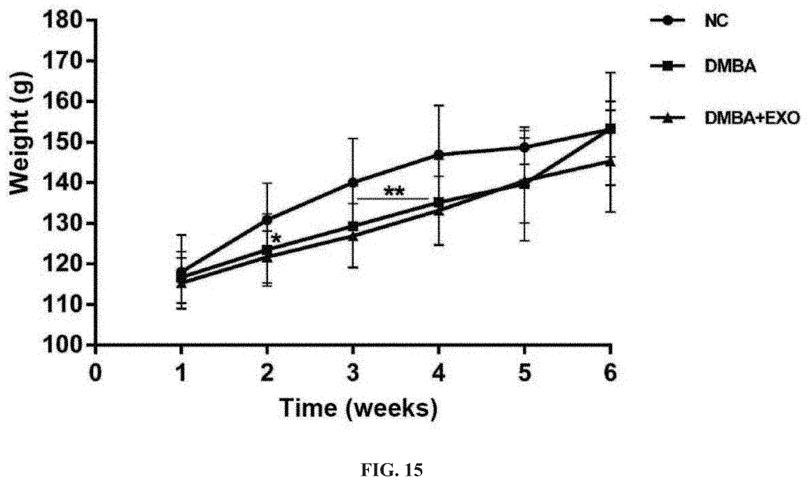

(2) Weight: at the beginning of the experiment, the body weight of each group was close. In the second, third and fourth weeks, the DMBA group and DMBA+EXO group were in acute inflammatory stage due to the sustained application of DMBA on cheek pouch, which affected eating and slowed weight gain. Body weight was significantly different from the negative control group. Precancerous pathological lesion stage emerged after 4 weeks, during which the mucosa thickened and roughened, which had no effect on eating, and the weight of the three groups gradually approached (see FIG. 15).

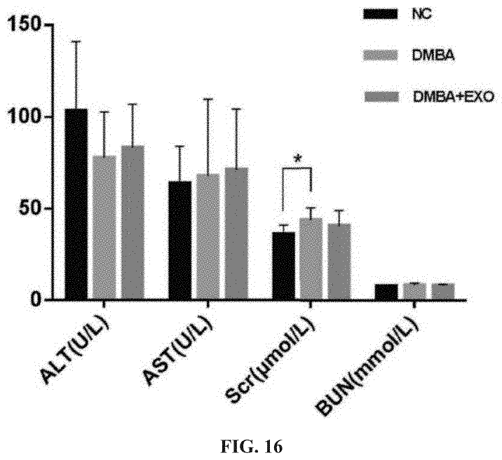

(3) Liver and kidney functions: by statistical analysis, there was no difference in ALT, AST, BUN of the biochemical markers related to liver and kidney functions in the serum of the four groups of hamsters. The level of Scr was higher in the DMBA group and the difference was statistically significant, suggesting that the hamsters of DMBA group might have a certain degree of kidney function damage. There was no statistical difference in the four indexes between DMBA+EXO group and negative control group, which further confirmed that exosomes, as a natural liposome, had good tolerance, stability and non-toxicity in vivo, and were ideal drug carriers (see FIG. 16).

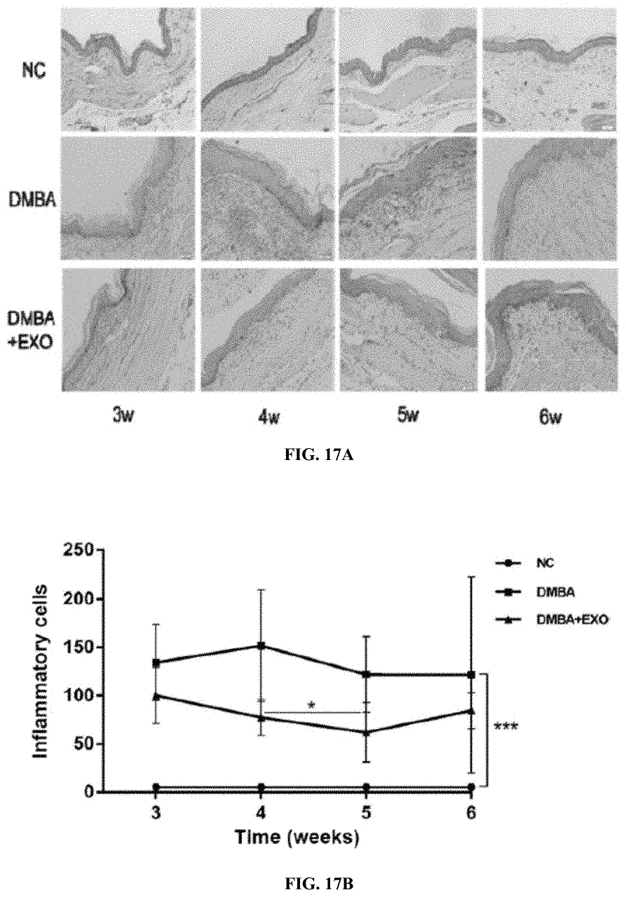

(4) Inflammatory cell expression and counting: From the second week to the end of the third week of the experiment was the acute inflammatory stage. It gradually transformed to precancerous lesions accompanied by chronic inflammation at the beginning of the fourth weekdue to the continuous action of DMBA (see FIG. 17A). Statistical analysis showed that the number of inflammatory cells in DMBA+EXO group at each stage significantly decreased compared with the DMBA group. The difference was significant at the end of four and five weeks (see FIG. 17B), confirming that topical application of exosomes carrying miR-185 inhibited local inflammation of the mucosa.

(5) Simple hyperplasia and abnormal hyperplasia counts: the statistical analysis of simple hyperplasia counts showed that there was no difference between the DMBA group and the DMBA+EXO group, and the treatment did not effectively reduce the number of simple hyperplasia.

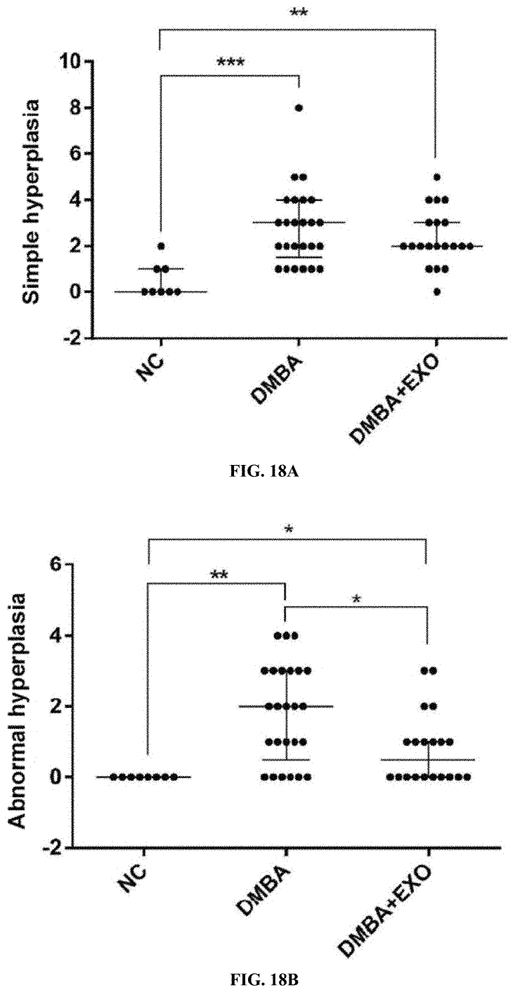

Abnormal hyperplasia counts showed that the DMBA+EXO group was significantly lower than the DMBA group, and the treatment delayed the conversion of lesions from simple to abnormal hyperplasia, effectively reduced the number of abnormal hyperplasia, and blocked the development of precancerous lesions. The results are shown in the table below and FIGS. 18A-B.

| Groups | n | Simple hyperplasia | Abnormal hyperplasia |
|---|---|---|---|
| NC | 8 | 0 (0-1) ^{#} | 0 (0-0) ^{#} |
| DMBA | 25 | 3 (2-4) | 2 (1-3) |
| DMBA + EXO | 20 | 2 (2-3) | 0.5 (0-1) * |

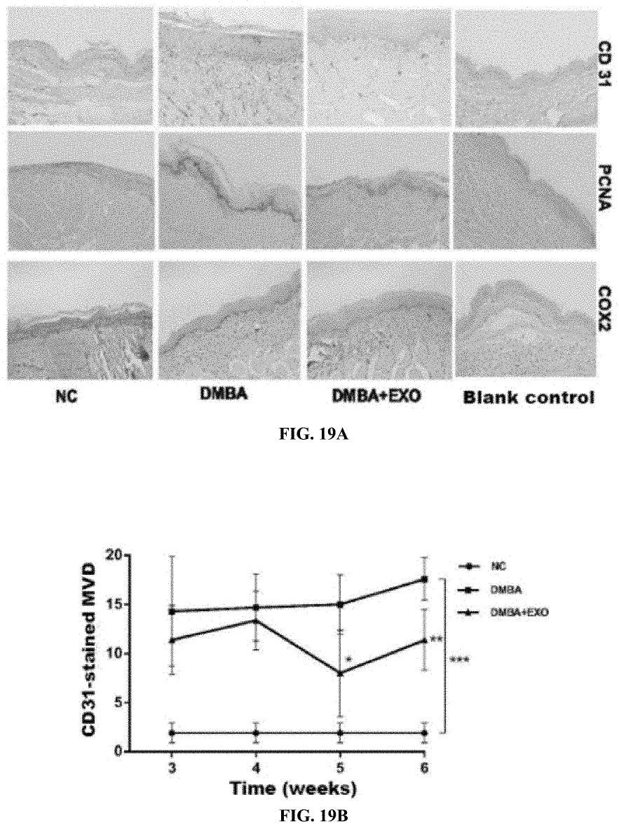

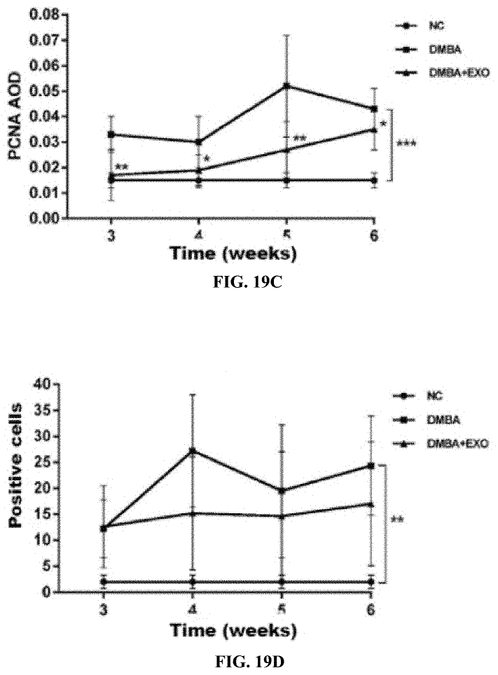

(6) Analysis of CD31, PCNA and COX2 expressions: the expressions of CD31, PCNA, COX2 are shown in FIG. 19A. The results of microvascular density (MVD) values calculated by CD31-labeled vascular endothelial cells showed that the DMBA+EXO group was lower than the DMBA group at each stage. The difference between the ends of fifth and sixth weeks was statistically significant (see FIG. 19B), confirming that topical application of exosomes carrying miR-185 had a good effect on inhibiting mucosal microvascular formation. The calculated epithelial AOD values after PCNA staining showed that the DMBA+EXO group was significantly lower than the DMBA group at each stage (see FIG. 19), confirming that topical application of exosomes carrying miR-185 had a good effect on inhibiting epithelial proliferation, which was also consistent with the results of abnormal hyperplasia counts. The positive cells count by COX2 staining showed that the DMBA+EXO group was basically the same as the DMBA group at the end of third week. The treatment group was lower than the DMBA group at the end of fourth, fifth, and sixth weeks, and the difference was not significant (see FIG. 19D), confirming that topical application of exosomes carrying miR-185 had a certain inhibitory effect on mucosal inflammation.

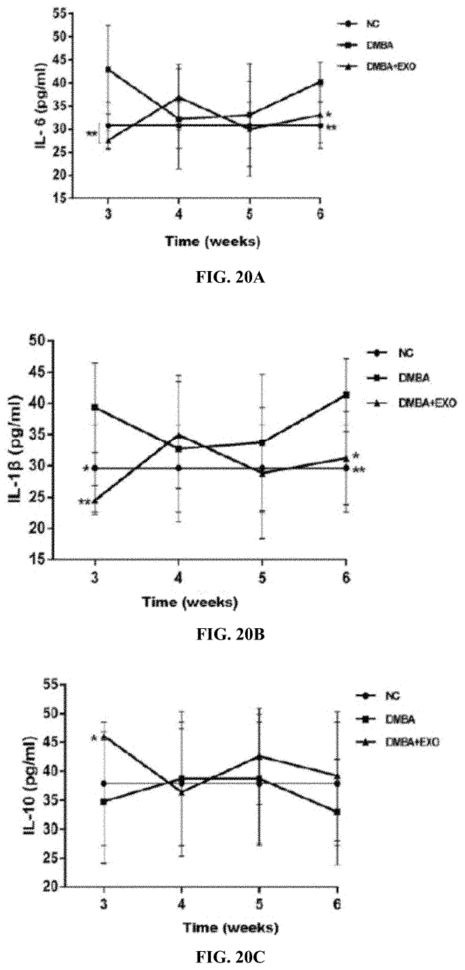

(7) Serum IL-1β, IL-6, IL-10 enzyme-linked immunosorbent assay: the expression levels of proinflammatory factors IL-1β, IL-6 were highly consistent in all stages and groups. The DMBA+EXO group was significantly lower than the DMBA+EXO group at the end of the third week acute inflammatory stage and the sixth week precancerous lesions stage. The expression levels were slightly fluctuated in the fourth and fifth weeks, and there was no statistical difference (see FIG. 20A, B). The anti-inflammatory factor IL-10 was significantly higher in the DMBA+EXO group than in the DMBA group at the end of the third week acute inflammatory stage, and the level was slightly fluctuated in the fourth week. The DMBA+EXO group was still higher than the DMBA group in the fifth and sixth weeks, which had no statistical difference (see FIG. 20). We found that the expression of IL-10, an inflammatory precursor, was synergistic with the expressions of proinflammatory factors IL-1(3 and IL-6, confirming that the serum inflammatory factors were significantly inhibited by local application of exosomes carrying miR-185.

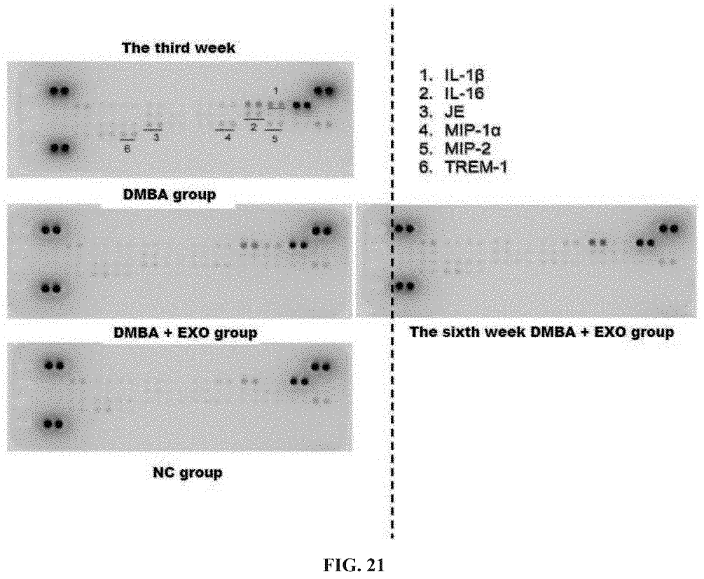

(8) Proteome Profiler Array Mouse Cytokine Array Panel A Assay: the results showed that the protein expressions of IL-1β, IL-16, TREM-1 and others in DMBA group increased when compared with those in DMBA+EXO group and negative control group at the end of the third week. The protein expressions of inflammatory factors in DMBA+EXO group did not change significantly at the end of the third and sixth weeks, and there was no significant difference between the DMBA+EXO group and the negative control group (see FIG. 21), confirming that topical application of exosomes carrying miR-185 had a good inhibition effect on mucosal inflammation.

### REFERENCES

1. Noguti J, De Moura C F, De Jesus G P, et al. Metastasis from oral cancer: an overview. Cancer Genomics Proteomics 2012: 9: 329-35.
2. Lingen M W, Pinto A, Mendes R A, et al. Genetics/epigenetics of oral premalignancy: current status and future research. Oral Dis 2011; 17Suppl 1: 7-22.
3. Nogami T, Kuyama K, Yamamoto H. Histopathological and immunohistochemical study of malignant transformation of oral leukoplakia, with special reference to apoptosis-related gene products and proliferative activity. Acta Otolaryngol 2003; 123: 767-75.
4. Thompson L. World Health Organization classification of tumours: pathology and genetics of head and neck tumours. Ear Nose Throat J 2006; 85: 74.
5. [symbol] [symbol] [symbol] [J/CD]. [symbol] [symbol] ), 2008, 2(5): 427-430.
6. Krisanaprakornkit S, Iamaroon A. Epithelial-mesenchymal transition in oral squamous cell carcinoma. ISRN Oncol 2012; 2012: 681469.
7. Mohtasham N, Babakoohi S, Salehinejad J, et al. Mast cell density and angiogenesis in oral dysplastic epithelium and low-and high-grade oral squamous cell carcinoma. Acta Odontol Scand 2010; 68: 300-4.
8. Chen Y K, Huse S S, Lin L M. Expression of inhibitor of apoptosis family proteins in human oral squamous cell carcinogenesis. Head Neck 2011; 33: 985-98.
9. Nomura H, Uzawa K, Yamano Y, et al. Overexpression and altered subcellular localization of autophagy-related 16-like lin human oral squamous-cell carcinoma: correlation with lymphovascular invasion and lymphnode metastasis. Hum Pathol 2009; 40: 83-91.
10. Lame L, Bellacosa A. Epithelial—mesenchymal transition in development and cancer: role of phosphatidylinositol 3′ kinase/AKT pathways. Oncogene2005; 24: 7443-54.
11. von Zeidler S V, de Souza Botelho T, Mendonca E F, et al. E-cadherin as a potential biomarker of malignant transformation in oral leukoplakia: a retrospective cohort study. BMC Cancer 2014; 14: 972.
12. Chaw S Y, Majeed A A, Dalley A J, et al. Epithelial to mesenchymal transition (EMT) biomarkers—E-cadherin, beta-catenin, APC and Vimentin—in oral squamous cell carcinogenesis and transformation. Oral Oncol 2012; 48: 997-1006.
13. Scanlon C S, Van Tubergen E A, Inglehart R C, et al. Biomarkers of epithelial-mesenchymal transition in squamous cell carcinoma. J Dent Res 2013; 92: 114-21.
14. Katsuno Y, Lamouille S, Derynck R. TGF-beta signaling and epithelial-mesenchymal transition in cancer progression. Curr Opin Onco12013; 25: 76-84.
15. Fantozzi A, Gruber D C, Pisarsky L, et al. VEGF-mediated angiogenesis links EMT-induced cancer stemness to tumor initiation. Cancer Res2014; 74: 1566-75.
16. Denton D, Xu T, Kumar S. Autophagy as a pro-death pathway. Immunol Cell Biol 2015; 93: 35-42.
17. White E. The role for autophagy in cancer. J Clin Invest 2015; 125: 42-6.
18. Akalay I, Janji B, Hasmim M, et al. Epithelial-to-mesenchymal transition and autophagy induction in breast carcinoma promote escape from T-cell-mediated lysis. Cancer Res 2013; 73: 2418-27.
19. Qin W, Li C, Zheng W, et al. Inhibition of autophagy promotes metastasis and glycolysis by inducing ROS in gastric cancer cells. Oncotarget 2015; 6: 39839-54.
20. Peng Y F, Shi Y H, Ding Z B, et al. Autophagy inhibition suppresses pulmonary metastasis of HCC in mice via impairinganoikis resistance and colonization of HCC cells. Autophagy 2013; 9: 2056-68.
21. Zhu G, He Y, Yang S, et al. Identification of Gene and MicroRNA Signatures for Oral Cancer Developed from Oral Leukoplakia. Biomed Res Int2015; 2015: 841956.
22. Bartel D P. MicroRNAs: genomics, biogenesis, mechanism, and function. Cell 2004; 116: 281-97.
23. Cervigne N K, Reis P P, Machado J, et al. Identification of a microRNA signature associated with progression of leukoplakia to oral carcinoma. Hum Mol Genet 2009; 18: 4818-29.
24. Gorenchtein M, Poh C F, Saini R, et al. MicroRNAs in an oral cancer context-from basic biology to clinical utility. J Dent Res 2012; 91: 440-6.
25. Wu B H, Xiong XP, Jia J, et al. MicroRNAs: new actors in the oral cancer scene. Oral Oncol 2011; 47: 314-9.
26. Li J, Huang H, Sun L, et al. MiR-21 indicates poor prognosis in tongue squamous cell carcinomas as an apoptosis inhibitor[J]. ClinCancer Res, 2009, 15 (12): 3998-4008. doi: 10.1158/1078-0432. CCR-08-3053.
27. Liu X, Wang A, Heidbreder C E, et al. MicroRNA-24 targeting RNAbinding protein DND1 in tongue squamous cell carcinoma[J]. FEBS Lett, 2010, 584(18): 4115-4120. doi: 10.1016/j.febslet. 2010. 08. 040.
28. Lu Y C, Chang J T, Liao C T, et al. OncomiR-196 promotes an invasive phenotype in oral cancer through the NME4-JNK-TIMP1-MMP signaling pathway[J]. Mol Cancer, 2014, 13: 218. doi: 10.1186/1476-4598-13-218.
29. Tiwari A, Shivananda S, Gopinath K S, et al. MicroRNA-125a reduces proliferation and invasion of oral squamous cell carcinoma cells by targeting estrogen-related receptor alpha: IMPLICATIONS FOR CANCER THERAPEUTICS [J]. J Biol Chem, 2014, 289 (46): 32276-32290. doi: 10.1074/jbc.M114.584136.
30. Chang C J, Hsu C C, Chang C H, et al. Let-7d functions as novel regulator of epithelial-mesenchymal transition and chemoresistant property in oral cancer[J]. Oncol Rep, 2011, 26(4): 1003-1010. doi: 10. 3892/or. 2011. 1360.
31. Kim J S, Park S Y, Lee S A, et al. MicroRNA—205 suppresses the oral carcinoma oncogenic activity via down-regulation of Axin-2 in KB human oral cancer cell[J]. Mol Cell Biochem, 2014, 387 (1-2): 71-79, doi: 10.1007/s11010-013-1872-7.
32. Momen-Heravi F, Trachtenberg A J, Kuo W P, et al. Genomewide study of salivary MicroRNAs for detection of oral cancer[J]. J Dent Res, 2014, 93 (7suppl): 86S-93S. doi: 10.1177/0022034514531018.
33. Hong S H, Ondrey F G, Avis I M, et al. Cyclooxygenase regulates human oropharyngeal carcinomas via the proinflammatory cytokine IL_6: a general role for inflammation? [J]FASEB J, 2000, 14(11): 1499-1507).
34. Hyde N C, Prvulovich E, Newman L, et al, A new approach to pretreatment assessment of the NO in oral squamous cell carcinoma: the role of sentinel node biopsy and emission tomography. Oral Oncol, 200, 39: 350-360.
35. Brow A E, Langdon J D. Management of oral cancer. Ann R Coll Surg Engl, 1995, 77: 404-408.

1. A method for prophylaxis or prevention of transformation from oral leukoplakia to oral cancer comprising administering to a leukoplakia subject a therapeutically effective amount of an exosome carrying miR-185.

2. The method of claim 1, wherein the leukoplakia is leukoplakia with simple hyperplasia or leukoplakia with abnormal hyperplasia.

3. The method of claim 1, wherein the oral cancer is oral squamous cell carcinoma.

4. The method of claim 1, wherein the exosome carrying miR-185 is administered together with other drugs or methods used for prevention of the transformation from oral leukoplakia to oral cancer.

5. A method for treatment of oral leukoplakia comprising administering to a leukoplakia subject a therapeutically effective amount of an exosome carrying miR-185.

6. The method of claim 5, wherein the leukoplakia is leukoplakia with simple hyperplasia or leukoplakia with abnormal hyperplasia.

7. The method of claim 5, wherein the treatment comprises reducing an area of leukoplakia or eliminating leukoplakia, or alleviating abnormal hyperplasia of leukoplakia, reversing to simple hyperplasia, or transforming leukoplakia to normal mucosa.

8. The method of claim 5, wherein the exosome carrying miR-185 is administered together with other drugs or methods used for treatment of oral leukoplakia.

9-12. (canceled)

13. The method of claim 1, wherein the exosome carrying miR-185 is administered to a subject by a topical route of administration.

14. The method of claim 1, wherein the exosome carrying miR-185 is administered to the subject by submucosal injection, topical smear, or buccal administration.

15. A method for prophylaxis of oral cancer, comprising administering to a subject a prophylactically effective amount of an exosome carrying miR-185, wherein the exosome prevents the transformation of simple mucosal leukoplakia to leukoplakia with abnormal hyperplasia and oral cancer or prevents the transformation of leukoplakia with abnormal hyperplasia to oral cancer by one or more of the following mechanisms: inhibition of inflammation response, inhibition of oral mucosal epithelial cell abnormal hyperplasia, and inhibition of mucosal microangiogenesis.

16. (canceled)

17. A modified salivary exosome introduced with a prophylactically or therapeutically effective amount of miR-185.

18. A pharmaceutical composition for prophylaxis or prevention of the transformation of oral leukoplakia to oral cancer comprising the modified salivary exosome of claim 17.

19. The composition of claim 18, wherein the leukoplakia is leukoplakia with simple hyperplasia or leukoplakia with abnormal hyperplasia.

20. The composition of claim 18, the oral cancer is oral squamous cell carcinoma.

21. A kit or pharmaceutical product comprising the exosome carrying miR-185 of claim 17.

22-24. (canceled)

25. A method for inhibiting the proliferation of oral cancer cells, comprising administering to a subject an effective amount of miR-185 or an exosome carrying miR-185 to inhibit the growth of oral cancer cells.

26. A method for regulating expression of oral cancer cell-associated proteins VEGF and AKT in a subject with oral cancer, comprising administering an effective amount of miR-185 or an exosome carrying miR-185 to the subject.

27. The method of claim 26, wherein the regulation includes inhibiting the expression of oral cancer cell-associated proteins VEGF and AKT.
